(19) United States
(12) Patent Application Publication
(10) Pub. No.: US 2005/0096490 A1
(43) Pub. Date: May 5, 2005

(54) Purification of perfluoromethane

(76) Inventors: Philip Bruce Henderson, Allentown, PA (US); Charles Gardner Coe, Macungie, PA (US)

Correspondence Address: AIR PRODUCTS AND CHEMICALS, INC.;PATENT DEPARTMENT, 7201 HAMILTON BOULEVARD, ALLENTOWN, PA, 181951501, US

(21) Appl. No.: 10/696,529

(22) Filed: Oct. 29, 2003

(51) Int. Cl. C07C 17/38

(52) U.S. Cl. 570/179

(57) ABSTRACT

Method for removing perfluoroethane (C₂F₆) from a gas mixture comprising perfluoroethane and one or more other fluorocarbon compounds, which method comprises contacting the gas mixture with an adsorbent comprising zeolite of the mordenite structure wherein at least a portion of the C₂F₆ is selectively adsorbed by the adsorbent, and withdrawing a C₂F₆-depleted gas product from contact with the adsorbent.

### BACKGROUND OF THE INVENTION

Perfluoromethane or tetrafluoromethane (CF₄) is an important material used in the manufacture of electronic components and may be used as a low-temperature refrigerant. In one method for the commercial production of CF₄, fluorine is reacted directly with carbon to yield a crude product comprising CF₄, unreacted fluorine, and reaction byproducts such as hydrofluoric acid (HF) and higher fluorinated alkyl compounds such as hexafluoroethane (C₂H₆) that result from the incomplete reaction of carbon with F₂. The residual F₂ and byproduct HF are removed by wet or dry scrubbing to yield impure CF₄ containing up to 1 vol % perfluoroethane (C₂F₆). Further purification is required to provide a final CF₄ product and the concentration of C₂F₆ for the highest purity CF₄ grades typically must be reduced to less than 0.5 parts per million by volume (ppmv).

Mixed fluorocarbons may be separated or purified by pressure swing or temperature swing adsorption processes. For example, U.S. Pat. No. 5,417,742 discloses adsorption processes utilizing silicon-rich zeolite adsorbents to recover perfuorocarbons from low-boiling gases such as nitrogen, oxygen, argon, and mixtures thereof. U.S. Pat. No. 5,523,499 discloses a method for purifying hexafluoroethane containing CClF₃ and/or CHF₃ impurities by adsorption on activated carbon or zeolite adsorbents. U.S. Pat. Nos. 5,785,741 and 6,187,077 describe combined membrane-adsorption processes for recovering fluorocarbons from mixtures of nitrogen, NF₃, SF₆, and fluorocarbons such as CF₄, CHF₃, and C₂F₆.

There is a need in the art for improved adsorption processes for removing C₂F₆ from gas mixtures containing fluorocarbons, and particularly for removing C₂F₆ as an impurity from CF₄. There is a need for adsorbents having a high adsorption capacity for adsorbed impurities, thereby allowing a long onstream time for a given adsorber size, and also having a high selectivity between the impurities and the CF₄ product. Such improved adsorbents, which can enhance the recovery of CF₄, are provided by embodiments of the present invention as disclosed and claimed below.

### BRIEF SUMMARY OF THE INVENTION

Embodiments of the invention described herein include a method for removing perfluoroethane (C₂F₆) from a gas mixture comprising perfluoroethane and one or more other fluorocarbon compounds, which method comprises contacting the gas mixture with an adsorbent comprising zeolite of the mordenite structure wherein at least a portion of the C₂F₆ is selectively adsorbed by the adsorbent, and withdrawing a C₂F₆-depleted gas product from contact with the adsorbent. The zeolite adsorbent may have a silica to alumina (Si/Al) atomic ratio of less than about 50. The zeolite adsorbent may be decationized such that at least about 50% of the cations are replaced with protons.

In one embodiment, one of the one or more other fluorocarbon compounds in the gas mixture may be perfluoromethane (CF₄). The C₂F₆-depleted gas product may comprise CF₄. The C₂F₆-depleted gas product may comprise at least 99.99 vol % CF₄.

The method for removing perfluoroethane (C₂F₆) from a gas mixture containing perfluoroethane and one or more other fluorocarbon compounds may comprise a pressure swing adsorption process using at least the steps of
(a) introducing the gas mixture at a feed pressure into an adsorber vessel containing the zeolite adsorbent of the mordenite structure having a silicon to aluminum (Si/Al) atomic ratio of less than about 50, selectively adsorbing at least a portion of the C₂F₆ on the adsorbent, and withdrawing the C₂F₆-depleted gas product from the adsorber vessel;
(b) terminating flow of the gas mixture into the adsorber vessel and reducing the pressure in the adsorber vessel by withdrawing a C₂F₆-enriched waste gas therefrom, thereby desorbing C₂F₆ and regenerating the adsorbent;
(c) pressurizing the adsorber vessel; and
(d) repeating steps (a) through (c) in a cyclic manner.

The pressure swing adsorption process may be carried out using two or more adsorber vessels operating out of phase such that one adsorber vessel undergoes steps (b) and (c) while another vessel undergoes step (a). At least a portion of the C₂F₆-enriched waste gas withdrawn from one adsorber vessel undergoing step (b) may be introduced into another adsorber vessel undergoing step (c).

When one of the one or more other components in the gas mixture is perfluoromethane (CF₄), the method may further comprise purging the adsorber vessel with an inert gas during step (b) to provide a purge gas effluent comprising the inert gas, C₂F₆, and CF₄, passing the purge gas effluent through secondary adsorber vessel in which C₂F₆ is selectively adsorbed, withdrawing a secondary purge stream comprising purge gas and CF₄ from the secondary adsorber, cooling and partially condensing purge stream to provide a cooled purge stream, and recovering condensed CF₄ from the cooled purge stream.

Alternatively, the method for removing perfluoroethane (C₂F₆) from a gas mixture containing perfluoroethane and one or more other fluorocarbon compounds may comprise a temperature swing adsorption process using at least the steps of
(a) introducing the gas mixture at a feed temperature into an adsorber vessel containing the zeolite adsorbent of the mordenite structure having a silicon to aluminum (Si/Al) atomic ratio of less than about 50, selectively adsorbing at least a portion of the C₂F₆ on the adsorbent, and withdrawing the C₂F₆-depleted gas product from the adsorber vessel;
(b) terminating flow of the gas mixture into the adsorber vessel, purging the vessel with a purge gas at a temperature greater than the feed temperature, and withdrawing from the vessel a purge gas effluent comprising the inert gas and C₂F₆, thereby desorbing C₂F₆ and regenerating the adsorbent;
(c) cooling the adsorber vessel; and
(d) repeating steps (a) through (c) in a cyclic manner.

The temperature swing adsorption process may be carried out using two or more adsorber vessels operating out of phase such that one adsorber vessel undergoes steps (b) and (c) while another vessel undergoes step (a). At least a portion of step (b) may be carried out in a temperature range of 50° C. to 300° C. The purge gas may be selected from the group consisting of nitrogen, argon, helium, hydrogen, and mixtures thereof.

When one of the one or more other components in the gas mixture is perfluoromethane (CF₄), the method may further comprise cooling the purge gas effluent to provide a cooled purge gas effluent containing the inert gas, C₂F₆, and CF₄, passing the cooled purge gas effluent through secondary adsorber vessel in which C₂F₆ is selectively adsorbed, withdrawing a secondary purge stream comprising purge gas and CF₄ from the secondary adsorber, cooling and partially condensing purge stream to provide a cooled purge stream, and recovering condensed CF₄ from the cooled purge stream.

Another embodiment of the invention is a high purity perfluoromethane (CF₄) product obtained by the steps comprising
(a) providing a gas mixture comprising CF₄ and at least perfluoroethane (C₂F₆);
(b) contacting the gas mixture with a zeolite adsorbent of the mordenite structure having a silicon to aluminum (Si/Al) atomic ratio of less than about 50 wherein at least a portion of the C₂F₆ is selectively adsorbed by the adsorbent to provide gas enriched in CF₄; and
(c) withdrawing the gas enriched in CF₄ from contact with the adsorbent to provide the high purity CF₄ product.

The zeolite adsorbent may be decationized such that at least about 50% of the cations are replaced with protons. The high purity CF₄ product may comprise at least 99.99 vol % CF₄.

### DETAILED DESCRIPTION OF THE INVENTION

Embodiments of the present invention are directed to improved methods of removing perfluoroethane (C₂F₆) from mixtures containing C₂F₆, other fluorocarbons, and optionally NF₃. One embodiment of the invention relates to an improved method for removing C₂F₆ from perfluoromethane (CF₄) to produce a high purity CF₄ product. In this improved method, zeolite of the mordenite (MOR) structure in the acid form (H⁺MOR) may be used in pressure or temperature swing adsorption processes to remove C₂F₆ as an impurity.

Mordenite is a naturally-occurring aluminosilicate mineral that is also produced synthetically in commercial quantities. The mordenite structure consists of aluminosilicate chains that are crosslinked by the sharing of neighboring oxygens. The building units of mordenite consist of four- and five-membered rings wherein each of the framework tetrahedrons belong to at least one of the five-membered rings in the structure. The principle feature of the mordenite structure is the presence of a main channel having dimensions of about 6.7×7.0 Angstrom wherein the main channel is parallel to the c-crystallographic axis and is accessible to gas molecules through a 12-ring. Side pockets with dimensions of about 5.7×2.9 Angstrom, which are perpendicular to the main channel along the b-crystallographic axis, are accessible through 8-rings. These side pockets provide limited access to the secondary pore system contained within the mordenite structure. The side pockets are spaced at regular intervals along the main channel and provide a “rough” surface in the main channel for molecules too large to diffuse through the small pore side pockets into the next main channel.

In the development work supporting the present invention, mordenite was found unexpectedly to possess higher selectivity for C₂F₆ than other common commercially available zeolites, thereby providing an improved adsorbent for removing C₂F₆ from mixtures of C₂F₆ with other fluorocarbons. In one embodiment, it was found in particular that certain mordenites are well-suited for removing C₂F₆ from CF₄ and have favorable equilibrium and kinetic properties which allow high recovery of CF₄ from regeneration purge gas.

Synthetic mordenite typically contains at least one metal cation and naturally-occuring mordenite typically contains a variety of metal cations. These metal cations may be replaced readily with acidic protons using conventional ion exchange processes. The ion exchange may be carried out in either the solid state or the liquid phase. The resulting mordenite in the acid or decationized form may be identified as H⁺MOR. In the present invention, at least about 50% of the cations in the original mordenite preferably are replaced with protons by ion exchange to yield H⁺MOR.

The Si/Al atomic ratio of mordenite may be varied over a wide range during synthesis or through a variety of post-synthesis modifications. All of these mordenite compositions may be converted readily to a predominantly acid form as described above, and in some cases this conversion process may alter the Si/Al atomic ratio of the mordenite. In the present invention, the Si/Al atomic ratio of the mordenite is preferably less than about 50 and most preferably less than about 10.

Purification of CF₄ derived from the direct fluorination of carbon may be effected by temperature swing adsorption using an adsorbent containing the H⁺MOR zeolite. CF₄ typically is co-adsorbed on the H⁺MOR zeolite, and this adsorbed portion may be lost when the bed temperature is raised and the bed is purged or evacuated to remove the adsorbed impurities. In order to recover some of the co-adsorbed CF₄ product, the spent adsorbent bed may be purged with nitrogen wherein the CF₄, which is less strongly adsorbed than the C₂F₆, desorbs into the purge gas. The purge gas, containing desorbed CF₄ and small amounts of desorbed C₂F₆, may be passed through a second bed of H⁺MOR zeolite until the C₂F₆ concentration rises to an unacceptable level in the CF₄/N₂ purge gas effluent stream. A significant amount, typically greater than 50%, of the co-adsorbed CF₄ may be recovered in this manner before the desorbing C₂F₆ concentration becomes too high. The CF₄ may be recovered from the purge effluent gas by cooling the gas, condensing the CF₄, and recovering the condensed CF₄.

In contrast with the acid mordenite (H⁺MOR), it was found that non-mordenite zeolites in the acid form did not show enhanced selectivity for C₂F₆. The advantages of H⁺MOR over other types of zeolite are given in the following Examples, which illustrate embodiments of the present invention but do not limit the invention to any of the specific details described therein.

### EXAMPLE 1

272 g of H⁺MOR(CP504-11CY, Zeolyst) having a Si/Al atomic ratio of 6 was packed into a 1 inch diameter column with a length of 3 ft. The effluent of the column was analyzed for percent concentration of CF₄ and ppm concentration of C₂F₆ by gas chromatography (GC). The column was saturated with CF₄ and allowed to reach a constant temperature. A gas mixture containing 1000 ppmv of C₂F₆ in CF₄ was passed through the column at 100 sccm while the effluent from the column was analyzed every 3 minutes. The breakthrough time was defined as the time 3 ppmv of C₂F₆ was detected by GC. After 14.5 hours, C₂F₆ breakthrough was observed, and this corresponds to a C₂F₆ adsorbent capacity of 0.014 mmole/g at breakthrough.

### EXAMPLE 2

Directly after breakthrough of C₂F₆, the column from Example 1 was purged at ambient temperature with a flow of 100 sccm N₂ while analyzing the purity of the desorbed CF₄. The desorbed CF₄ in the N₂ could be recovered by passing the mixture through a second activated carbon column to remove the C₂F₆ and separated from the purge N₂ cryogenically. During purge and desorption of the CF₄ from the H⁺MOR, a constant level of 25 ppm of C₂F₆ was observed in the purge gas effluent. After 45 minutes, the CF₄ concentration in the N₂ dropped below 10% and purge was terminated.

### EXAMPLE 3

The experiment described in Example 1 was repeated with 278 g of type NaX zeolite in place of the H⁺MOR. C₂F₆ breakthrough was observed after 13.0 hours and this corresponded to a C₂F₆ capacity of 0.012 mmole/g.

### EXAMPLE 4

A CF₄ recovery experiment similar to Example 2 was performed on the column from Example 3. After 60 minutes the CF₄ concentration in the N₂ dropped below 10% and desorption was stopped. At the start of the desorption of CF₄, the C₂F₆ concentration was observed at 60 ppm and steadily rose to 285 ppm at the end of the recovery step.

Comparing Examples 1 and 3, H⁺MOR is shown to have 16% more capacity for C₂F₆ compared to NaX zeolite on a mmole/g basis. Since packing densities are about the same for the two adsorbents, H⁺MOR will allow for on-stream times >15% longer for removing C₂F₆ from CF₄. A comparison of Examples 2 and 4 show that CF₄ is more easily recovered from purge gas with H⁺MOR compared with NaX and a significantly lower amount of impurities are desorbed during the CF₄ recovery step than with NaX. Moreover, during the desorption of the CF₄, the C₂F₆ concentration was low and constant on H⁺MOR compared to the steady increasing levels using NaX.

### EXAMPLE 5

The breakthrough experiment in Example 1 was repeated with 242 g of the zeolite adsorbent HZSM-5. After only 9 hours, breakthrough of C₂F₆ occurred, and this corresponded to a loading of 0.010 mmoles/g at breakthrough. Because of its inferior C₂F₆ adsorption performance, the desorption experiment was not carried out for HZSM-5. The lower C₂F₆ selectivity for the HZSM-5 led to a shorter breakthrough time, showing that the acid form of other zeolites does not enhance the purification of CF₄ beyond that achievable with standard NaX adsorbent.

In addition to the packed bed breakthrough studies described above, standard equilibrium isotherm measurements were carried out on several zeolite adsorbents. Isotherm measurements were made at 70° C. on the adsorbents after careful activation under dynamic vacuum to 400° C. to ensure that no structural damage occurred from the activation. Henry's law constants were obtained from the adsorption isotherms and were used to calculate the Henry's law selectivities for C₂F₆ relative to CF₄ for the adsorbents studied. Selectivity is defined herein as the ratio of the Henry's law constant for C₂F₆ to the Henry's law constant for CF₄. These selectivities are given below in Table 1.

TABLE 1
Equilibrium Henry's Law C₂F₆/CF₄ selectivities at 70° C.

| | | | Selectivity |
|---|---|---|---|
| Adsorbent | Source | Si/Al atomic ratio | C₂F₆/CF₄ |
| NaX pellets | UOP Corp | 1.2 | 12.3 |
| LiLSX | Zeochem | 1.0 | 7.7 |
| NaMordenite | Zeolyst | 5 | no adsorption |
| H Mordenite | Zeolyst | 6 | 28.4 |
| H Mordenite | Zeolyst | 10 | 24.4 |
| H Mordenite | Zeolyst | 45 | 20.2 |
| CaMordenite | standard aqueous | 10 | no adsorption |
| | Ca exchange | | |
| HZSM-5 | Zeolyst | 150 | 9.8 |
| HTON (ZSM-22) | Zeolyst | Not measured | 4.0 |
| HMTT (ZSM-23) | Zeolyst | Not measured | 2.2 |
| MCM-41 | synthesized | >1000 | 2.2 |

From the results in Table 1 it is seen that the acid form of mordenite (H⁺MOR) possesses unique sorption properties for selectively removing C₂F₆ from CF₄. Based on some early results for H⁺MOR compared to the NaX sorbent, it was believed that the selectivity of the H⁺MOR could be increased by increasing the charge density of the sorption space as well as the amount of volumetric micropore volume. An initial attempt was made to increase the charge density by replacing the acidic proton with sodium. The addition of sodium totally blocked access to the zeolite for both CF₄ and C₂F₆ and resulted in no measurable adsorption as seen above for NaX. An attempt to “open up” the zeolite's access by using divalent calcium ions in place of the monovalent sodium ions was also unsuccessful.

Since Na mordenite is a one-dimensional zeolite having relatively low micropore volume, an attempt was made to use an X-type zeolite known to have larger pore size as well as a higher volumetric density. The X-type adsorbent chosen was LiLSX, which has the highest density of high charge density lithium cations and is the best known sorbent available for O₂ VSA applications. Unexpectedly, the C₂F₆/CF₄ selectivity for LiLSX was actually less than that found for the standard NaX adsorbent. This result suggested that the cation type is not a strong influence on the selectivity.

Based on these results, the influence of pore size and framework composition on the selectivity were explored. Two zeolites having the ZSM-5 structure were tested to see if smaller pore size (5.5 Angstrom vs. 6.5 Angstrom) or the presence of five rings in the zeolite structure would influence the selective adsorption of C₂F₆. A highly siliceous ZSM-5 having less than one Al per unit cell was found to give inferior selectivities to those of HMOR. A mesoporous silica, MCM-41, which had 40 Angstrom pores giving further reduction in the electric field gradient present in the channels, also was tested. This sorbent was the most non-selective evaluated and showed a C₂F₆/CF₄ selectivity of only 2.2.

The acid forms of other zeolites having similar structural features to those of mordenite also were evaluated. Two one-dimensional structures having smooth walls and ten-ring channels, generally referred to as ZSM-22 and ZSM-23, were evaluated and were expected to be better than H⁺MOR since ZSM-22 and ZSM-23 had similar porosity and smaller channels to give higher affinity for C₂F₆ relative to CF₄. The results indicated that neither of these zeolites was superior to the H⁺MOR.

A final series of experiments explored the influence of aluminum content in the mordenite structure on the C₂F₆/CF₄ selectivity. Three different mordenites with Si/Al ratios ranging from 6 to 45 were evaluated. As shown in Table 1, increasing the aluminum content had a positive influence on the C₂F₆/CF₄ selectivity, and there appears to be a unique feature of the mordenite structure that contributes to the enhanced C₂F₆/CF₄ selectivity. One possible reason for the enhanced selectivity may be the presence of the side pocket, which may have the proper shape to “capture” C₂F₆ molecules. Another possible reason may be the corrugated walls present in the mordenite channel giving molecular specificity to the solid/gas interactions dependent on molecular shape and size. These are clearly unexpected results that distinguish mordenite-containing adsorbents as preferred materials for removing C₂F₆ from bulk CF₄.

The preferred adsorbent described above may be used in a pressure swing, temperature swing, or pressure and temperature swing adsorption process to remove more strongly adsorbed C₂F₆ from a gas mixture that also contains at least one less strongly adsorbed component. The adsorbent particle diameter may be between 0.5 and 3.0 mm. The feed temperature may be in the range of 0 to 100° C. and the feed pressure may be in the range of 1 to 20 atm abs. In one embodiment, the gas mixture may comprise CF₄ containing C₂F₆ as a minor constituent in the range of 500 to 5000 ppmv and the process may be used to recover a high purity CF₄ product.

A pressure swing adsorption process for removing the C₂F₆ from the gas mixture that also contains at least one less strongly adsorbed component typically includes multiple cyclic steps. In a first step, the gas mixture at a feed pressure is introduced into an adsorber vessel containing the zeolite adsorbent described above, preferably a zeolite of the mordenite structure having a silica to alumina ratio of less than 50. At least a portion of the C₂F₆ is selectively adsorbed on the adsorbent and a C₂F₆-depleted gas product is withdrawn from the adsorber vessel. After a selected period of time, the flow of the gas mixture into the adsorber vessel is terminated and the pressure in the adsorber vessel is reduced by withdrawing a C₂F₆-enriched waste gas therefrom, thereby desorbing C₂F₆ and regenerating the adsorbent. The adsorber vessel is pressurized with the feed gas mixture and optionally with another compatible gas. The steps are then repeated in a cyclic manner.

The pressure swing adsorption process may be carried out using two or more adsorber vessels operating out of phase such that one adsorber vessel undergoes steps regeneration and pressurization while another vessel undergoes the feed step. In another process option, the pressure swing adsorption process may be carried out using two or more adsorber vessels operating out of phase such that at least a portion of the C₂F₆-enriched waste gas withdrawn from one adsorber vessel undergoing depressurization is introduced into another adsorber vessel undergoing the pressurization step, thereby at least partially repressurizing the vessel. This optional step will improve the recovery of the C₂F₆-depleted gas product.

One of the less strongly adsorbed components in the gas mixture may be perfluoromethane (CF₄). In this embodiment, the process may further comprise purging the adsorber vessel with an inert gas during the regeneration step at approximately ambient temperature and 0.1 to 2 atm abs to provide a purge gas effluent comprising the inert gas, C₂F₆, and CF₄, passing the purge gas effluent through secondary adsorber vessel in which C₂F₆ is selectively adsorbed, withdrawing a secondary purge stream comprising purge gas and CF₄ from the secondary adsorber, cooling and partially condensing purge stream to provide a cooled purge stream, and recovering condensed CF₄ from the cooled purge stream.

Alternatively, a temperature swing adsorption process having multiple cyclic steps may be used for removing the C₂F₆ from the gas mixture that also contains at least one less strongly adsorbed component. In this process, the gas mixture at a feed temperature is introduced into an adsorber vessel containing the zeolite described above, preferably a zeolite adsorbent of the mordenite structure having a silica to alumina ratio of less than about 50. The zeolite adsorbent may be decationized such that at least about 50% of the cations are replaced with protons.

At least a portion of the C₂F₆ is selectively adsorbed on the adsorbent, and a C₂F₆-depleted gas product is withdrawn from the adsorber vessel. The flow of the gas mixture into the adsorber vessel is terminated and the vessel is purged with a purge gas at a temperature greater than the feed temperature, for example, at a temperature in the range of 50° C. to 300° C. and a pressure in the range of 0.1 to 2 atm abs. The purge gas may be selected from the group consisting of nitrogen, argon, helium, hydrogen, and mixtures thereof. A purge gas effluent comprising the inert gas and C₂F₆ is withdrawn from the vessel, thereby desorbing C₂F₆ and regenerating the adsorbent. The adsorber vessel is cooled and the steps are repeating in a cyclic manner.

This temperature swing adsorption process may be carried out using two or more adsorber vessels operating out of phase such that one adsorber vessel undergoes regeneration while another vessel undergoes the feed step. In one embodiment, the gas mixture may contain perfluoromethane (CF₄) and the purge gas effluent may be cooled to provide a cooled purge gas effluent containing the inert gas, C₂F₆, and CF₄. A high purity CF₄ product containing at least 99.99 vol % CF₄ is produced in this embodiment. The cooled purge gas effluent may be passed through a secondary adsorber vessel in which C₂F₆ is selectively adsorbed and a secondary purge stream comprising purge gas and CF₄ may be withdrawn from the secondary adsorber. The purge stream may be cooled and partially condensing to provide a cooled purge stream, and condensed CF₄ may be recovered from the cooled purge stream.

1. A method for removing perfluoroethane (C₂F₆) from a gas mixture comprising perfluoroethane and one or more other fluorocarbon compounds, which method comprises contacting the gas mixture with an adsorbent comprising zeolite of the mordenite structure wherein at least a portion of the C₂F₆ is selectively adsorbed by the adsorbent, and withdrawing a C₂F₆-depleted gas product from contact with the adsorbent.

2. The method of claim 1 wherein the zeolite adsorbent has a silicon to aluminum (Si/Al) atomic ratio of less than about 50.

3. The method of claim 1 wherein the zeolite adsorbent is decationized such that at least about 50% of the cations are replaced with protons.

4. The method of claim 1 wherein one of the one or more other fluorocarbon compounds in the gas mixture is perfluoromethane (CF₄).

5. The method of claim 4 wherein the C₂F₆-depleted gas product comprises CF₄.

6. The method of claim 5 wherein the C₂F₆-depleted gas product comprises at least 99.99 vol % CF₄.

7. The method of claim 1 comprising a pressure swing adsorption process using at least the steps of
(a) introducing the gas mixture at a feed pressure into an adsorber vessel containing the zeolite adsorbent of the mordenite structure having a silicon to aluminum (Si/Al) atomic ratio of less than about 50, selectively adsorbing at least a portion of the C₂F₆ on the adsorbent, and withdrawing the C₂F₆-depleted gas product from the adsorber vessel;
(b) terminating flow of the gas mixture into the adsorber vessel and reducing the pressure in the adsorber vessel by withdrawing a C₂F₆-enriched waste gas therefrom, thereby desorbing C₂F₆ and regenerating the adsorbent;
(c) pressurizing the adsorber vessel; and
(d) repeating steps (a) through (c) in a cyclic manner.

8. The method of claim 7 wherein the pressure swing adsorption process is carried out using two or more adsorber vessels operating out of phase such that one adsorber vessel undergoes steps (b) and (c) while another vessel undergoes step (a).

9. The method of claim 7 wherein the pressure swing adsorption process is carried out using two or more adsorber vessels operating out of phase such that at least a portion of the C₂F₆-enriched waste gas withdrawn from one adsorber vessel undergoing step (b) is introduced into another adsorber vessel undergoing step (c).

10. The method of claim 6 wherein one of the one or more other components in the gas mixture is perfluoromethane (CF₄) and wherein the method further comprises purging the adsorber vessel with an inert gas during step (b) to provide a purge gas effluent comprising the inert gas, C₂F₆, and CF₄, passing the purge gas effluent through secondary adsorber vessel in which C₂F₆ is selectively adsorbed, withdrawing a secondary purge stream comprising purge gas and CF₄ from the secondary adsorber, cooling and partially condensing purge stream to provide a cooled purge stream, and recovering condensed CF₄ from the cooled purge stream.

11. The method of claim 1 comprising a temperature swing adsorption process using at least the steps of
(a) introducing the gas mixture at a feed temperature into an adsorber vessel containing the zeolite adsorbent of the mordenite structure having a silicon to aluminum (Si/Al) atomic ratio of less than about 50, selectively adsorbing at least a portion of the C₂F₆ on the adsorbent, and withdrawing the C₂F₆-depleted gas product from the adsorber vessel;
(b) terminating flow of the gas mixture into the adsorber vessel, purging the vessel with a purge gas at a temperature greater than the feed temperature, and withdrawing from the vessel a purge gas effluent comprising the inert gas and C₂F₆, thereby desorbing C₂F₆ and regenerating the adsorbent;
(c) cooling the adsorber vessel; and
(d) repeating steps (a) through (c) in a cyclic manner.

12. The method of claim 11 wherein the temperature swing adsorption process is carried out using two or more adsorber vessels operating out of phase such that one adsorber vessel undergoes steps (b) and (c) while another vessel undergoes step (a).

13. The method of claim 11 wherein at least a portion of step (b) is carried out in a temperature range of 50° C. to 300° C.

14. The method of claim 11 wherein the purge gas is selected from the group consisting of nitrogen, argon, helium, hydrogen, and mixtures thereof.

15. The method of claim 11 wherein one of the one or more other components in the gas mixture is perfluoromethane (CF₄) and wherein the method further comprises cooling the purge gas effluent to provide a cooled purge gas effluent containing the inert gas, C₂F₆, and CF₄, passing the cooled purge gas effluent through secondary adsorber vessel in which C₂F₆ is selectively adsorbed, withdrawing a secondary purge stream comprising purge gas and CF₄ from the secondary adsorber, cooling and partially condensing purge stream to provide a cooled purge stream, and recovering condensed CF₄ from the cooled purge stream.

16. A high purity perfluoromethane (CF₄) product obtained by the steps comprising
(a) providing a gas mixture comprising CF₄ and at least perfluoroethane (C₂F₆);
(b) contacting the gas mixture with a zeolite adsorbent of the mordenite structure having a silicon to aluminum (Si/Al) atomic ratio of less than about 50 wherein at least a portion of the C₂F₆ is selectively adsorbed by the adsorbent to provide gas enriched in CF₄; and
(c) withdrawing the gas enriched in CF₄ from contact with the adsorbent to provide the high purity CF₄ product.

17. The product of claim 16 wherein the zeolite adsorbent is decationized such that at least about 50% of the cations are replaced with protons.

18. The product of claim 16 wherein the high purity CF₄ product comprises at least 99.99 vol % CF₄.
